(19) United States
(12) Patent Application Publication
(10) Pub. No.: US 2014/0180244 A1
(43) Pub. Date: Jun. 26, 2014

(54) LOCKING ASSEMBLY FOR PREVENTING DISPENSING OF DOSE FROM MEDICATION DISPENSING DEVICE

(71) Applicant: Eli Lilly and Company, Indianapolis, IN (US)

(72) Inventor: Jaime Ray Arnett, Fishers, IN (US)

(73) Assignee: Eli Lilly and Company, Indianapolis, IN (US)

(21) Appl. No.: 14/108,424

(22) Filed: Dec. 17, 2013

Related U.S. Application Data
(60) Provisional application No. 61/740,107, filed on Dec. 20, 2012.

(51) Int. Cl. A61M 5/315

(52) U.S. Cl. CPC A61M 5/31566; A61M 5/31501
USPC 604/506; 604/201

(57) ABSTRACT

A locking assembly for a medication dispensing device including a housing, a drive member, a medication cartridge having a barrel holding medication between a movable plunger and a septum, and a dose delivery mechanism for controlling the advancement of the drive member. The locking assembly, in one form, includes a lock member, biased to move from a first position to a second position, with a first actuating element. The locking assembly also includes a second actuating element disposed on the drive member and cooperatively configured with the first actuating element. If the drive member of the device moves rearward from the shipped position due to a freezing of the medication, the second actuating element engages the first actuating element such that the lock member moves from the first position to the second position to lock the medication dispensing device from further dose dispensing.

### BACKGROUND OF THE INVENTION

The present invention pertains to medication dispensing devices, and, in particular, to a locking assembly that prevents the medication dispensing device in which it is provided from delivering a dose if the medication contents have been frozen before the first use of the device.

Patients suffering from a number of different diseases frequently must inject themselves with medication. To allow a person to conveniently and accurately self-administer medicine, a variety of devices broadly known as injector pens or injection pens have been developed. Generally, these pens are equipped with a cartridge including a piston or plunger and containing a multi-dose quantity of liquid medication. A drive member, extending from within a base of the injector pen and operably connected with typically more rearward mechanisms of the pen that control drive member motion, is movable forward to advance the plunger in the cartridge in such a manner to dispense the contained medication from an outlet at the opposite cartridge end, typically through a needle that penetrates a stopper at that opposite end. In disposable pens, after a pen has been utilized to exhaust the supply of medication within the cartridge, the entire pen is discarded by a user, who then begins using a new replacement pen.

One problem that arises with some disposable dispensing devices is related to the fact their medication contents need to be refrigerated. From the time the disposable dispensing device is made by the manufacturer until the time the device is first used by the patient, such as during shipping or prolonged storage before being supplied to the patient, it is possible that the device may be subject to refrigeration conditions that actually result in the freezing of its cartridge medication. Such freezing may be a basis to recommend not using such medication. In at least one known device, such freezing, due to the expansion of the medication contents that causes the cartridge plunger to be driven backward and in so doing force the drive member backward as well, such freezing can compromise the proper operation of the device. While labels that indicate freezing could be used with these devices, such labels could be overlooked in which case the devices may be used by unknowing patients.

Thus, it would be desirable to provide a locking assembly that prevents a dose from being delivered from a medication dispensing device if the medication contents of the device have been frozen prior to the first use of the device by the patient.

### BRIEF SUMMARY OF THE INVENTION

In one form thereof, the present invention provides a locking assembly for a medication dispensing device including a housing, a drive member extending in an axial direction within the housing, a medication cartridge having a barrel holding medication between a movable plunger and a septum, the plunger engageable by the drive member within the barrel, and a dose delivery mechanism for controlling the advancement of the drive member forward in the axial direction to advance the plunger forward in the axial direction toward the septum for dispensing a medication dose when the septum is pierced by a needle. The locking assembly includes a lock member within the housing, the lock member including a first actuating element. The lock member is biased to move from a first position to a second position. The lock member, when in the second position, engages one of the drive member and the dose delivery mechanism to lock the medication dispensing device from further dose dispensing. The lock member, when in the first position, allows dose dispensing. The locking assembly also includes a second actuating element disposed on the drive member and cooperatively configured with the first actuating element. The second actuating element has a rearward end, in the axial direction, disposed between a rearward end of the first actuating element and the plunger. The second actuating element rearward end, when the device is initially shipped for patient use after manufacture, is in an axially spaced relationship with the first actuating element rearward end that is no more than an axial distance the drive member will be forced rearward from a shipped position if the medication expands upon freezing to drive the plunger rearward. The second actuating element, if the drive member moves rearward from the shipped position due to a freezing of the medication, engages the first actuating element such that the lock member moves from the first position to the second position to lock the medication dispensing device.

In another form thereof, the present invention provides a locking assembly for a medication dispensing device including a housing, a drive member extending in an axial direction within the housing, a medication cartridge having a barrel holding medication between a movable plunger and a septum, the plunger engageable by the drive member within the barrel, and a dose delivery mechanism for controlling the advancement of the drive member forward in the axial direction to advance the plunger forward in the axial direction toward the septum for dispensing a medication dose when the septum is pierced by a needle, the dose delivery mechanism including a component rotatable relative to the housing. The locking assembly includes a lock member within the housing, which lock member includes a first portion and a second portion extending from the first portion away from the plunger. The second portion includes a first locking element. The lock member is axially movable within the housing from a first position to a second position. The locking assembly also includes a second locking element disposed on the dose delivery mechanism component and cooperatively configured with the first locking element. The first locking element, when the lock member is disposed in the first position, is disengaged from the second locking element to allow rotation of the dose delivery mechanism component relative to the housing necessary for dose dispensing. The first locking element, when the lock member is disposed in the second position, engages the second locking element to prevent rotation of the dose delivery mechanism component relative to the housing to lock the medication dispensing device from further dose dispensing. The lock member, when the device is initially shipped for patient use after manufacture, is disposed within the housing in the first position. The lock member first portion is structured and arranged such that if the drive member moves rearward from the shipped position due to a freezing of the medication, the lock member first portion is contacted by the drive member to move the lock member from the first position to the second position to lock the medication dispensing device.

One advantage of the present invention is that a medication dispensing device can be provided with a mechanism for automatically locking the device to prevent administration of a dose if the contents of the device have been frozen prior to the first use of the device.

### BRIEF DESCRIPTION OF THE DRAWINGS

The above-mentioned and other advantages and objects of this invention, and the manner of attaining them, will become more apparent, and the invention itself will be better understood by reference to the following description of embodiments of the invention taking in conjunction with the accompanying drawings, wherein:

FIG. 1 is a top view of one type of medication dispensing device that is equipped with a first embodiment of a locking assembly of the present invention, which device is shown equipped with a needle assembly for injecting use;

FIG. 2 is an exploded perspective view of the medication dispensing device of FIG. 1, and with the needle assembly;

FIG. 3 is a top view in partial longitudinal cross-section of the medication dispensing device of FIG. 1 prior to the mounting of a needle assembly;

FIG. 4 is a partial diagrammatic side view, in partial longitudinal cross-section and with portions removed to better show the locking assembly, of the medication dispensing device of FIG. 3;

FIG. 5 is a partial diagrammatic side view similar to FIG. 4, further showing the arrangement of the locking assembly after activation caused by freezing of the cartridge medication;

FIGS. 6A, 6B and 6C are perspective views, and a side view, respectively, of the lock member shown separate from the remainder of the device of FIG. 1;

FIGS. 7A and 7B are a top view and a perspective view, respectively, of the drive member body shown separate from the remainder of the device of FIG. 1;

FIG. 8 is a partial view of a different type of medication dispensing device that is equipped with a second embodiment of a locking assembly of the present invention, which device and locking assembly are shown prior to cartridge medication freezing;

FIG. 9 is a perspective view of the lock member shown separate from the remainder of the device of FIG. 8; and

FIG. 10 is partial view similar to FIG. 8 further showing the arrangement of the locking assembly after activation caused by freezing of the cartridge medication.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

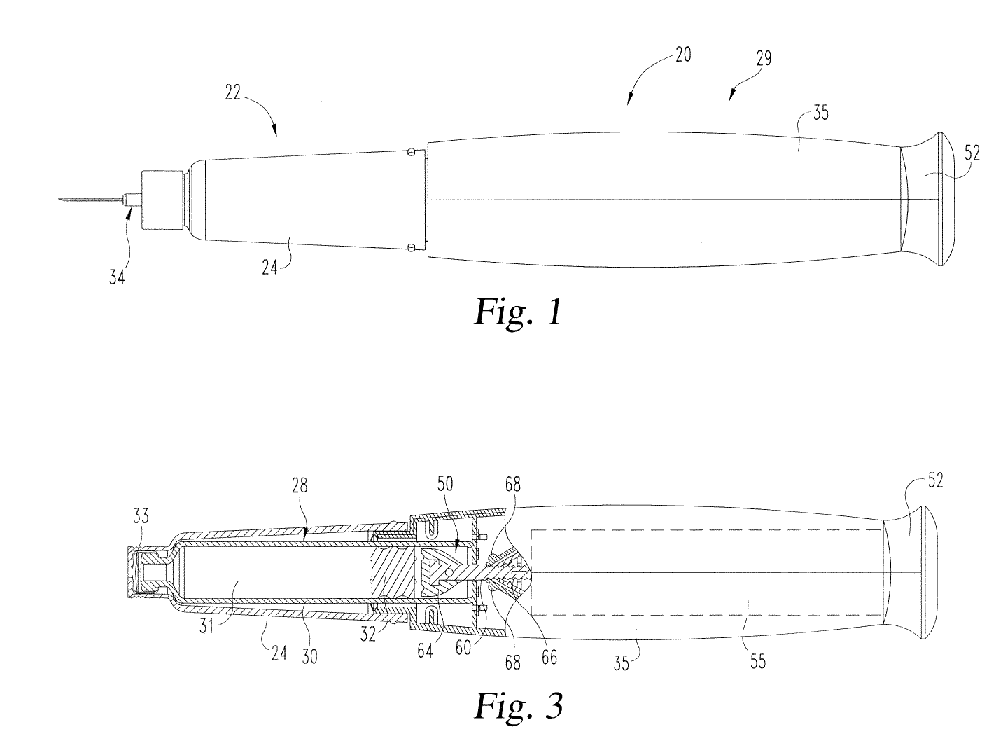

Referring now to FIGS. 1-3, there are shown various views of a disposable medication dispensing device or apparatus that advantageously has been provided with a first embodiment of a locking assembly of the present invention. The shown device, generally designated 20, is configured substantially the same as a device shown in U.S. Pat. No. 7,517,334, the entire contents of which are hereby incorporated by reference.

The locking assembly of the present invention advantageously may be incorporated into fixed or variable dose dispensing devices of various designs having a drive member that advances the cartridge plunger to force medication from the device, but which drive member is susceptible to being driven backward when the medication in the device is frozen. The mechanisms in such devices by which a dose is set or prepared and then delivered by advancement of the drive member is not relevant to the instant invention other than the fact that in some embodiments of the invention the locking assembly engages such mechanism, as opposed to engaging the drive member directly in another embodiment, to lock the device from dose delivery. Thus, the following description of device 20, other than the specifics of the locking assembly therein, is provided by way of background and intended to be illustrative and not limiting.

The distal portion 22 of the pen-shaped injection device 20 includes a clear retainer 24 that holds a cartridge 28 therein. Cartridge 28 is of conventional design, including a barrel 30 having an interior reservoir sealed at one end by a slidable plunger 32 and sealed at the other end by a septum 33. A needle assembly 34 shown mounted to retainer 24 pierces the septum 33 to provide an outlet during injection for the medication 31 filling the barrel reservoir, which medication 31 is intended to be delivered by operation of device 20.

The proximal portion 29 of device 20 includes a protective external housing 35 to which the retainer 24 is fixedly secured. Pen proximal portion 29 includes an axially advanceable drive member generally designated 50 within housing 35, an externally accessible control element 52 projecting from the proximal end of housing 35, and a force transfer assembly abstractly shown at 55 in FIG. 3. Drive member 50 is constrained by the interior surfaces of housing 35 to be axially translatable and rotatably fixed therein.

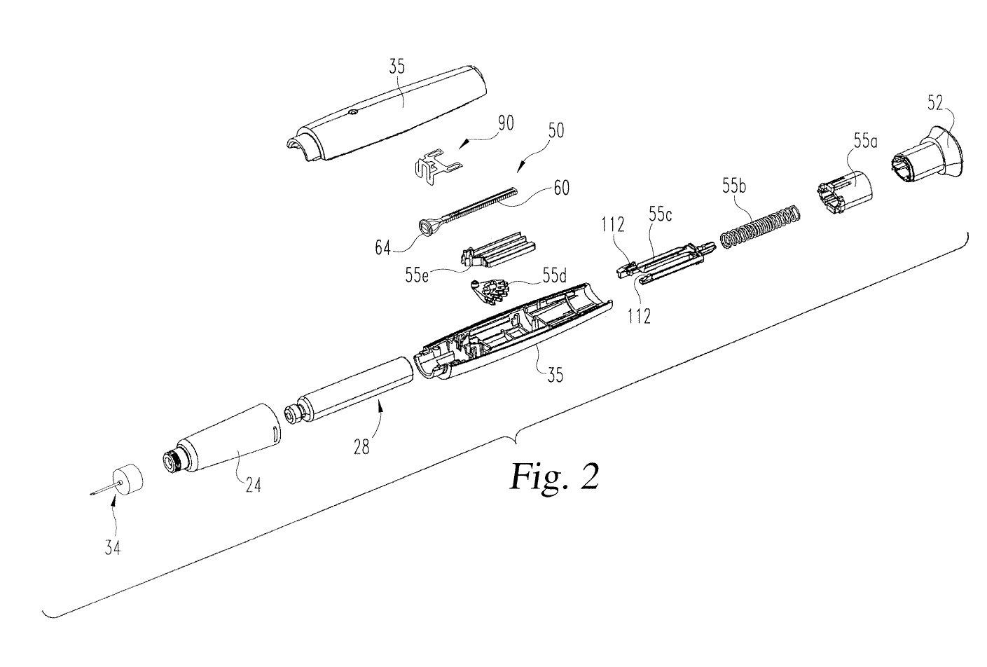

Force transfer assembly 55 operatively interconnects drive member 50 and control element 52. Force transfer assembly 55 is further shown in FIG. 2 as including a plunger piece 55a, a spring 55b, a plunger element 55c, a gear set 55d, and a gear-engaging piece 55e. When control element 52 is pulled from the position shown in FIG. 1 outward from the housing 35 to prepare the dose and then pushed back to the position shown in FIG. 1, control element 52 and force transfer assembly 55 together serve as a dose delivery mechanism to force the drive member 50 forward in the axial direction, or to the left in FIG. 3, to advance the cartridge plunger 32 forward to deliver the dose desired.

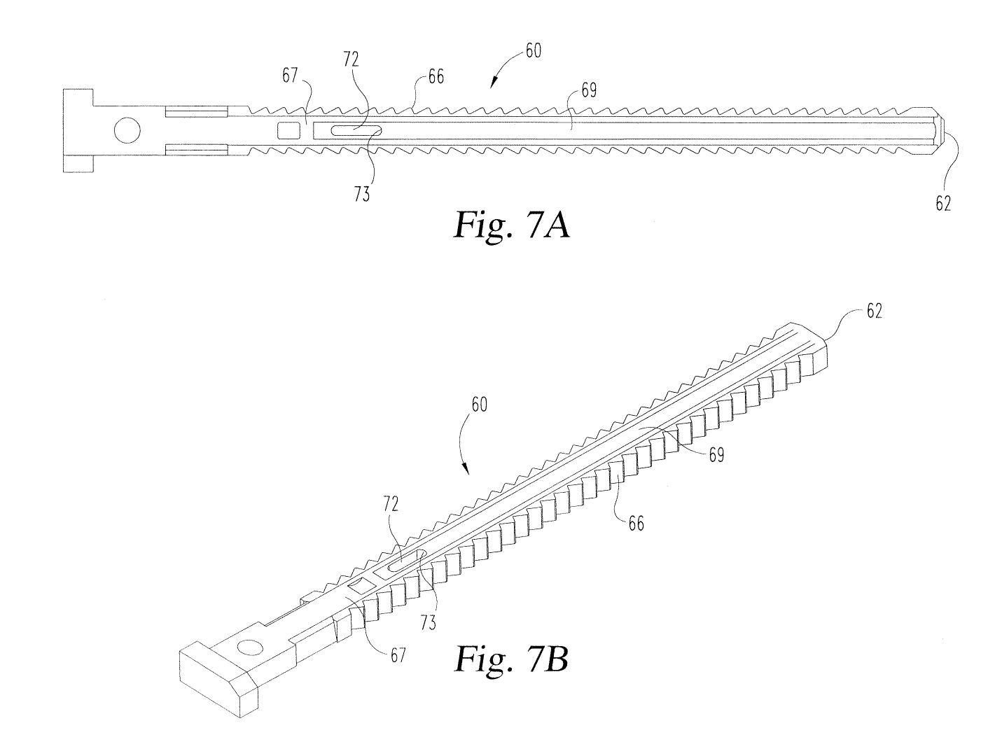

Drive member 50 includes a foot 64, and a rod-shaped body 60 that is generally rectangular in transverse cross-section. Foot 64 is formed at the distal end of body 60 and serves as a load distributing element on the plunger 32 that it directly engages. Body 60, shown separate in FIG. 7A and 7B, is made of metal and extends from foot 64 in the axial direction to a proximal end 62. Foot 64 may be provided as a plastic overmolding of the end of body 60.

A row of one-way ramping ratchet teeth 66 are formed on opposite sides of body 60. The teeth 66 continue uninterrupted along the axial length of the body extending from foot 64. Ratchet teeth 66 are engaged by a pair of diametrically opposed, resilient tabs or pawls 68 integrally formed with the housing. Pawls 68 slide along and over teeth 66 when drive member 50 is advanced distally within the housing during use, but abut the transverse, proximal face of teeth 66 to prevent drive member 50 from backing up in the proximal direction when subjected to normal loads. Pawls 68 are susceptible to breakage if drive member 50 is driven backward with sufficient force, such as may occur when the medication contents 31 of cartridge 28 freeze and thereby expand.

The opposite two sides of body 60 that are not provided with teeth 66 are designated 67. Each of the two sides 67 is contoured to provide a channel 69. Channel 69 serves as a guide in which slides the lock assembly skid 100. A channel 69 is provided on each of sides 67, despite skid 100 only engaging one of the sides, to allow drive member 50 to be installed in either of two rotational orientations to make manufacturing assembly less complicated. An elongate or slot-shaped opening 72 that extends completely through body 60 is provided near the distal end of channel 69. Opening 72 serves as a lock actuating element and is cooperatively configured with a complementary actuating element of a lock member. Opening 72 is designed to receive the skid 100 therein a sufficient depth for proper locking operation. Opening 72 is made with a longer axial length than skid 100, thereby allowing drive member 50 to move further rearward relative to skid after the device is locked. This longer axial length accounts for variation in displacement of the plunger 32 due to manufacturing volumetric variation of the medication 31, and for manufacturing variations in shapes and sizes of the component pieces, such as cartridge 28, drive member 50, housing 35, lock member 90 and force transfer assembly 55. Opening 72, rather than being a through hole as described, could instead be designed as a hollow in side 67 provided the skid penetration into such hollow is sufficient. Opening 72 is positioned along the axial length of body 60 in view of its function described further below.

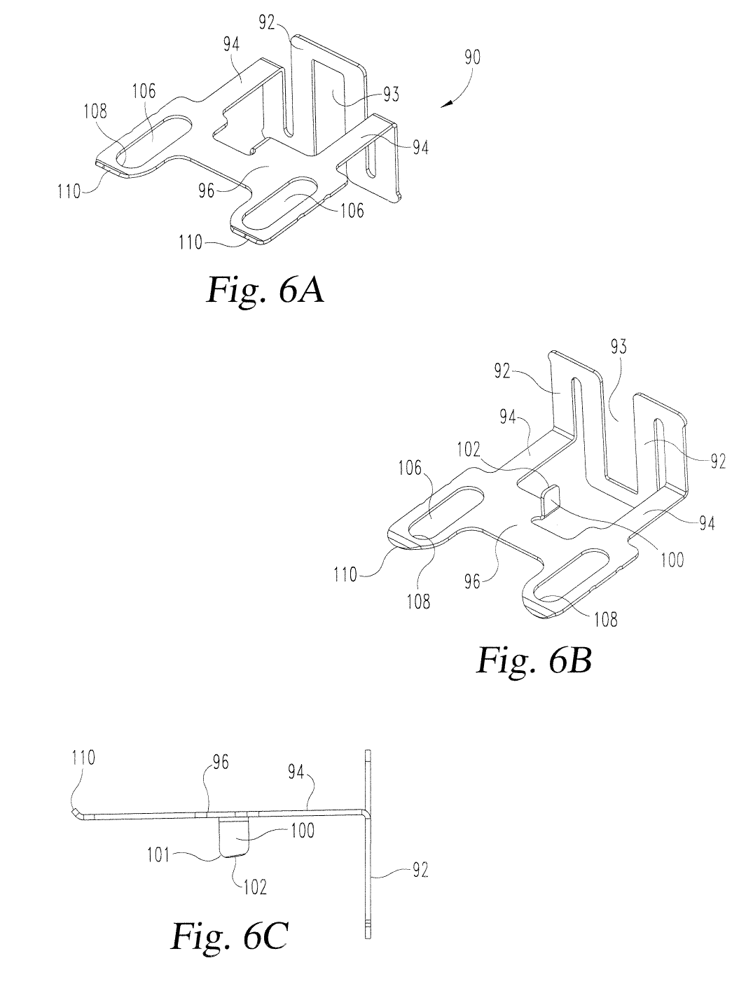

The locking assembly includes a lock or latch member, generally designated 90, shown further in FIGS. 6A, 6B and 6C. If not used due to freezing of the cartridge medication, the lock member 90 in the shown embodiment is used without opening 72 in the locking of the device after a final dose is administered as taught in U.S. Pat. No. 7,517,334. A suitable lock member for locking upon freezing could be a separate element from anything used in a final dose lock, or could be used in a device not having any final dose lock, within the scope of the present invention.

Lock member 90 is formed in a single piece, such as a metal stamping. Lock member 90 includes a multi-angled base flange 92 that defines an opening 93 through which drive member 50 freely passes. Base flange 92 is captured by complementarily shaped interior features of the housing 35 such that lock member 90 is axially fixed within the housing, and such that base flange 92 is also prevented from moving transverse to the axial direction. The opposite ends of flange 92 each have a spring arm 94 extending therefrom. Spring arms 94 are disposed orthogonally to flange 92 when in a neutral or non-stressed state.

A plate portion 96 of lock member 90 is located at the proximal ends of spring arms 94. Spring arms 94 are elastically bendable relative to the captured base flange 92 to allow plate portion 96 to be angled relative to base flange 92. Depending from plate portion 96 is a skid 100 that serves as an actuating element of the locking assembly. Skid 100 is flat and includes a bottom or lower end 102 that extends in the axial direction. Skid end 102 is angled relative to plate portion 96. This angling is designed such that the entire axial length of skid end 102 slides in contact with drive member 50 as the drive member advances during device use if the locking assembly is not actuated due to freezing prior to the first device use.

Plate portion 96 includes two generally elliptical eyelets 106 that form hook-contacting surfaces 108. Upturned edges 110 of plate portion 96 promote it being cammed over the hooks 112 associated with the plunger element 55c, which camming may be necessary when the lock member 90 is also intended to be able to lock device 20 after the administration of a final dose.

Skid 100 is of a height that its engagement with side 67 within channel 69 results in plate portion 96, due to bending of spring arms 94 which provides a returning biasing force, being directed or forced upward and away from its neutral position to a ready position, whereby the surfaces 108 are spaced from the plunger hooks 112 disposed thereunder so as to not interfere with device operation.

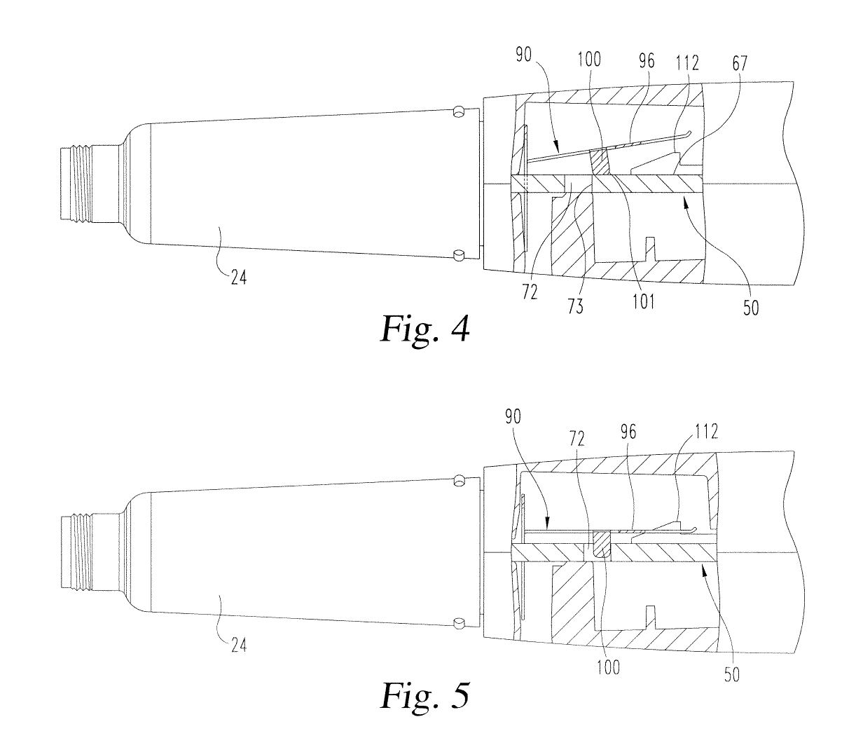

The structure of the locking assembly will be further understood in view of the following description of its operation in device 20. Device 20 is shown in FIGS. 3 and 4 arranged as when it is initially shipped for patient use after manufacture, whether it be for operation by a medical professional, the patient or another who administers to the patient. As the device is shipped without a needle assembly mounted to its end, the medication 31 does not have an outlet through the septum end of cartridge 28, and thus if medication 31 freezes it will tend to drive the plunger 32 rearward within cartridge 28 toward the drive member 50. In such shipped arrangement, as best shown in FIG. 4, the drive member 50 is axially positioned within the device housing 35 such that the rearward end 73 of slot-shaped opening 72 is in the axial direction disposed between the rearward end 101 of locking member skid 100 and cartridge plunger 32. The axially spaced relationship between the rearward ends of skid 100 and opening 72 is no more than an axial distance the drive member 50 will be forced rearward from the shown shipped position if the medication 31 fully freezes and expands to push the plunger 32 rearward.

If the medication 31 in device 20 does not freeze before its first use, the locking assembly of the present invention will not be activated, leaving lock member 90 in a ready position shown in FIG. 4 due to skid 100 engaging drive member side 67, allowing device 20 to be operated to dispense doses of medication in the ordinary manner.

On the other hand, if medication 31 fully freezes, its expansion will drive plunger 32 rearward. Plunger 32, as it moves rearward, will force drive member 50 rearward against pawls 68 that may break or deform, allowing drive member 50 to continue moving rearward within housing 35. This drive member motion also may cause rearward movement of components of the dose delivery mechanism. When drive member 50 has moved sufficiently rearward, such that the rearward end 73 of the slot 72 has passed the rearward end 101 of skid 100, slot 72 has axially moved into position below the skid 100 that is cooperatively figured to engage the slot 72 by insertion therein. Spring arms 94, due to their resiliency, then automatically drive plate portion 96 downward, or transverse to the axial direction, as skid 100 fits into slot 72, which downward motion snaps eyelets 106 down over plunger hooks 112 into the arrangement shown in FIG. 5. The resulting engagement of plate surfaces 108 with hooks 112 prevents the externally accessible control element 52 from being retracted to set and then inject a dose. This motion of the plate portion 96 from the ready position to latched position thus locks device 20 from further dose dispensing.

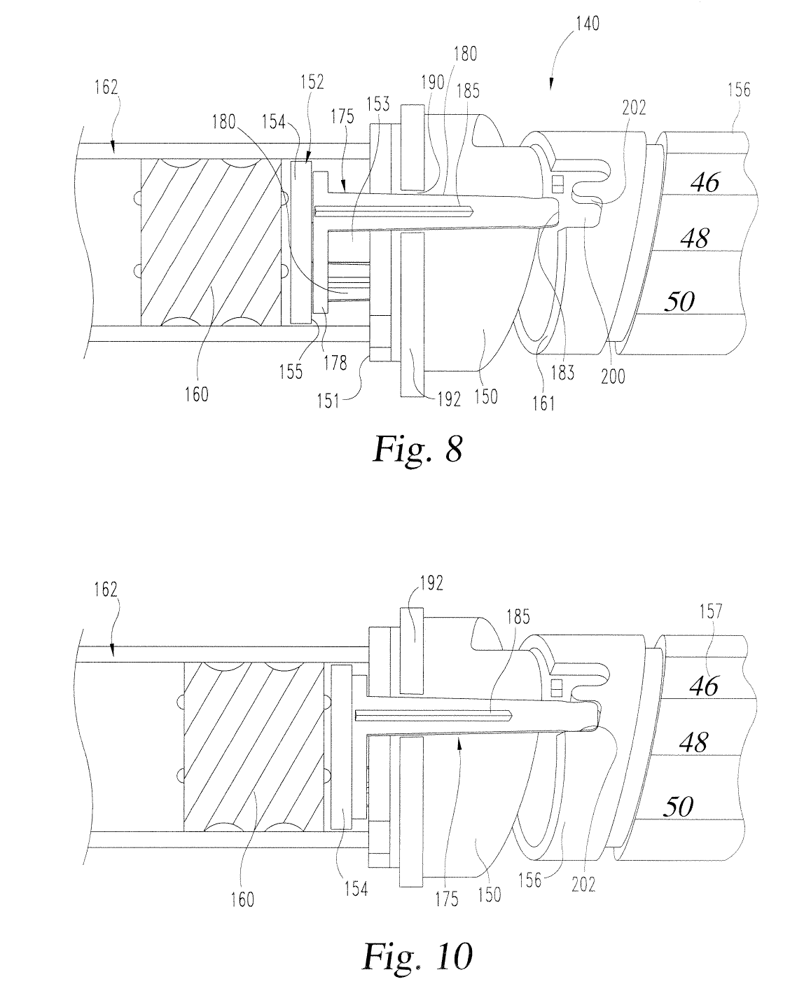

Referring now to FIGS. 8-10, there is shown an alternate embodiment of a locking assembly of the present invention. The locking assembly of FIGS. 8-10 is particularly suited for preventing a rotation of a rotatable component of a dose delivery mechanism of a device in which it installed so as to prevent medication being dispensing from that device if frozen before first device use.

One delivery device with which an alternate locking assembly finds beneficial application is shown in pertinent part in FIG. 8 and indicated generally as 140. Device 140 includes a housing bulkhead 150, an advanceable drive member generally designated 152, a sleeve 156 that is rotatable relative to bulkhead 150, and a medication filled cartridge 162 having a plunger 160. Bulkhead 150 has attached thereto the not shown external housing pieces that protectively cover the dose delivery mechanism, as well as the not shown retainer in which the medication cartridge 162 is held. Drive member 152 includes an axially extending body 153 and an enlarged diameter, disc-shaped foot 154 for engaging the cartridge plunger at the distal end of body 153. Sleeve 156 is part of the dose delivery mechanism of device 140 which is used to control the advancement of drive member 152 to deliver an intended dose. Sleeve 156 is shown as being a dose dial sleeve that is rotated out from the device housing to set a dose for delivery. Sleeve 156 has indicia 157 that in a conventional manner are visible through a not shown window in the not shown housing to indicate a set dose that device 140 is to inject when an externally accessible and not shown plunger button of the device is depressed. When that plunger button is so depressed, the drive member body 153 and foot 154 advance to the left in FIG. 8 to force cartridge plunger 160 to the left to force medication from cartridge 162 and out a not shown injection needle assembly mounted to the device.

The locking assembly of FIGS. 8-10 includes lock member 175 including a forward or distal portion 178 and a pair of rearwardly extending tabs 180. Lock member 175 may be formed as a one-piece metal stamping. Forward portion 178 is configured to directly engage the underside 155 of foot 154 when drive member 152 is driven proximally upon freezing of the cartridge contents prior to first use. Tabs 180 are configured to directly engage sleeve 156 as further described below.

Forward portion 178 is generally ring-shaped with a central, circular opening 184 through which drive member body 153 freely extends. The ring-shape of forward portion 178 provides for assured contact with foot 154 should it be needed, but different shapes, including one that does not ring the drive member body 153, may be used in alternate embodiments. Locking tabs 180 are integrally formed with and extend proximally from outward extending regions 179 formed in the outer radial periphery of forward portion 178. Although only a single tab could provide a locking function, a pair of tabs 180, spaced 180 degrees apart on forward portion 178, provide for load balancing. Tabs 180 extend within gaps 190 in a circumferential rib 192 of bulkhead 150.

Lock member 175 is maintained within device 140 in the ready position shown in FIG. 8 by the engagement of tabs 180 with the device housing. In particular, each tab 180 includes a longitudinally extending ridge 185 that projects radially outward. Ridges 185 provide an interference fit with the device external housing pieces by pressing tabs 180 inward against bulkhead 150. The interference fit provides a resistance to axial motion of lock member 175 from the ready position, but which resistance can be overcome when an axial force is applied to the distal face of forward portion 178 by foot 154 upon medication freezing. In an alternate embodiment, a maintaining element separate from tabs 180 may be used to hold lock member 175 within device 140 in its ready position.

When device 140 is initially shipped for patient use after manufacture, the lock member 175 is arranged in the ready position shown in FIG. 8. Axial space between forward portion 178 and the underside 155 of foot 154 when initially shipped may be provided so long as locking member 175 does not interfere with the dose delivery mechanism until it is to lock as intended. In this ready position, the underside of forward portion regions 179 are in axially spaced relationship with the distal end face 151 of bulkhead 150. This axial spacing is selected to be slightly greater than an axial distance the lock member 175 will be forced rearward by the drive member 152 when such drive member is forced rearward if the cartridge medication freezes before its first use.

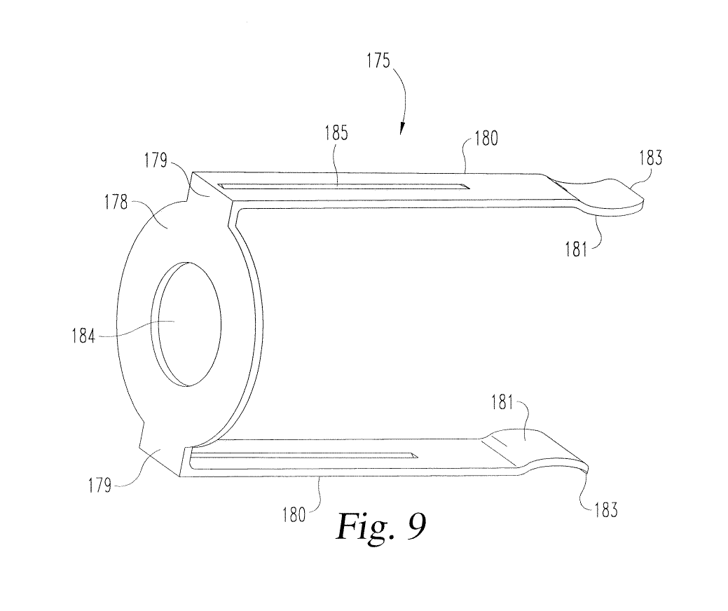

Tabs 180 are generally straight as they extend proximally from forward portion regions 179, but with a bending 181 at its proximal end 183 as shown in FIG. 9. Bending 181 produces an effectively radially thicker tab end 183 to increase locking engagement with the dose dial.

The locking features complementary to the locking feature served by tab ends 183 are curved surfaces 202 that define recesses 200 formed in the distal end 161 of sleeve 156 on diametrically opposite sides. Components other than the sleeve which experience rotation relative to the lock member 175 during device use may be provided with similar locking feature in alternate embodiments. When lock member 175 is disposed in the ready position, tab ends 183 are axially clear of sleeve end 161 as shown in FIG. 8 so as to not insert therein and thereby not interfere with the rotation of sleeve 156, allowing the dose delivery mechanism of device 140 to operate without interference.

If before the first use of device 140 the medication within cartridge 162 freezes causing plunger 160 to be forced rearward, the resulting proximal motion of drive member foot 154 results in lock member 175 being forced rearward. Such motion, which can continue until the underside of the outward extending regions 179 of forward portion 178 abut bulkhead end face 151, results in tab ends 183 being axially shifted into recesses 200, which is the locked arrangement of the locking assembly shown in FIG. 10. Tab ends 183, when inserted within recesses 200, prevent rotation of sleeve 156 by directly abutting surfaces 202 upon user attempts to rotate the drive sleeve 156, thereby preventing the dose delivery mechanism from operating and effectively locking the device 140 to prevent dose dispensing. A user will recognize the locking of the device as the drive sleeve locking will prevent a dose from even being set for device 140. The interference fit provided by ridges 185 with the housing still exists when lock member 175 is in the locked arrangement, such that once lock member 175 is so moved to that arrangement, it will not move axially forward even if the cartridge medication thaws and the foot 154 is able to move forward. Thus, the device 140 will remain locked.

Medication dispensing devices 20 and 140, and more particularly a device including a locking assembly claimed in this application, may be utilized in injecting a variety of medications or therapeutics into a person in need thereof so long as the locking assembly has not locked the device from dose delivery. The cartridge 28, 162 can be filled with a therapeutic such as teriparatide, which teriparatide when injected, for example, treats postmenopausal women with osteoporosis who are at high risk for fracture, or increases bone mass in men with primary or hypogonadal osteoporosis who are at high risk for fracture. Devices 20 and 140 may then be operated to inject a person with an effective amount of teriparatide. Therefore, for example, a method of treating a postmeopausal woman with osteoporosis by injecting teriparatide in the woman with a device having a locking assembly of the present invention is provided.

While this invention has been shown and described as having preferred designs, the present invention may be modified within the spirit and scope of this disclosure. For example, provided the skid of the embodiment of FIGS. 1-7 were made sufficiently robust, the plate portion that engages the plunger hooks could be dispensed with as the skid itself could directly lock the drive member from advancement. In which case, while the control element 52 could be pulled out to set a dose, it could not be pressed in because the drive member could not move forward. Still further, rather than the biasing feature for the lock member being integrated therein via its spring arms, a separate spring could be used to provide a biasing force. This application is therefore intended to cover any variations, uses or adaptations of the invention using its general principles. Further, this application is intended to cover such departures from the present disclosure as come within known or customary practice in the art to which this invention pertains.

I claim:

1. A locking assembly for a medication dispensing device including a housing, a drive member extending in an axial direction within the housing, a medication cartridge having a barrel holding medication between a movable plunger and a septum, the plunger engageable by the drive member within the barrel, and a dose delivery mechanism for controlling the advancement of the drive member forward in the axial direction to advance the plunger forward in the axial direction toward the septum for dispensing a medication dose when the septum is pierced by a needle, the locking assembly comprising:
a lock member within said housing, said lock member including a first actuating element, said lock member biased to move from a first position to a second position, said lock member, when in said second position, engaging one of the drive member and the dose delivery mechanism to lock the medication dispensing device from further dose dispensing, said lock member, when in said first position, allowing dose dispensing;
a second actuating element disposed on the drive member and cooperatively configured with said first actuating element, said second actuating element having a rearward end, in the axial direction, disposed between a rearward end of said first actuating element and the plunger, said second actuating element rearward end, when the device is initially shipped for patient use after manufacture, being in an axially spaced relationship with said first actuating element rearward end that is no more than an axial distance the drive member will be forced rearward from a shipped position if the medication expands upon freezing to drive the plunger rearward;
whereby said second actuating element, if the drive member moves rearward from the shipped position due to a freezing of the medication, engages the first actuating element such that said lock member moves from said first position to said second position to lock the medication dispensing device.

2. The locking assembly of claim 1 wherein said second actuating element comprises one of a hole and a hollow in an axially extending body of said drive member.

3. The locking assembly of claim 2 wherein said first actuating element comprises a skid that slides along a skid-engaging surface of the drive member and that engages said second actuating element by inserting within said hole or hollow.

4. The locking assembly of claim 1 wherein said lock member engages the dose delivery mechanism to lock the medication dispensing device.

5. The locking assembly of claim 1 wherein said first actuating element is structured and arranged to be said lock member that engages the drive member to lock the medication dispensing device.

6. A locking assembly for a medication dispensing device including a housing, a drive member extending in an axial direction within the housing, a medication cartridge having a barrel holding medication between a movable plunger and a septum, the plunger engageable by the drive member within the barrel, and a dose delivery mechanism for controlling the advancement of the drive member forward in the axial direction to advance the plunger forward in the axial direction toward the septum for dispensing a medication dose when the septum is pierced by a needle, the dose delivery mechanism including a component rotatable relative to the housing, the locking assembly comprising:
a lock member within said housing, said lock member including a first portion and a second portion extending from said first portion away from the plunger, said second portion including a first locking element, said lock member axially movable within the housing from a first position to a second position;
a second locking element disposed on the dose delivery mechanism component and cooperatively configured with said first locking element;
said first locking element, when said lock member is disposed in said first position, being disengaged from said second locking element to allow rotation of the dose delivery mechanism component relative to the housing necessary for dose dispensing;
said first locking element, when said lock member is disposed in said second position, engaging said second locking element to prevent rotation of the dose delivery mechanism component relative to the housing to lock the medication dispensing device from further dose dispensing;
said lock member, when the device is initially shipped for patient use after manufacture, disposed within said housing in said first position;
said lock member first portion structured and arranged such that if the drive member moves rearward from the shipped position due to a freezing of the medication, said lock member first portion is contacted by the drive member to move said lock member from said first position to said second position to lock the medication dispensing device.

7. The locking assembly of claim 6 wherein said first locking element comprises at least one bent tab end, and said second locking element comprises at least one recess-defining surface formed in the dose delivery mechanism component.

8. The locking assembly of claim 6 wherein said lock member first portion comprises a ring-shape for engaging a foot of the drive member and having a central opening through which an axially extending body of the drive member freely extends.

9. The locking assembly of claim 8 wherein said lock member second portion comprises a tab that extends rearward in the axial direction from a radial periphery of said first portion.

10. A method of treating a postmenopausal woman with osteoporosis by injecting teriparatide in the woman with a device having the locking assembly of claim 1.

11. A method of treating a postmenopausal woman with osteoporosis by injecting teriparatide in the woman with a device having the locking assembly of claim 6.
